(19) United States
(12) Patent Application Publication
(10) Pub. No.: US 2005/0265906 A1
(43) Pub. Date: Dec. 1, 2005

(54) Apparatus and Method for Preventing Radioactive Contamination of Radiopharmaceutical Manufacturing Components

(75) Inventor: Ahmad Najafi, Henderson, NV (US)

Correspondence Address: SQUIRE, SANDERS & DEMPSEY L.L.P, 600 HANSEN WAY, PALO ALTO, CA, 94304-1043, US

(73) Assignee: PALO ALTO SCIENTIFIC, INC., Henderson, NV (US)

(21) Appl. No.: 10/908,459

(22) Filed: May 12, 2005

Related U.S. Application Data
(60) Provisional application No. 60/576,006, filed on Jun. 1, 2004.

(51) Int. Cl. A61K 51/00; G06F 19/00; G01N 31/00; B32B 27/04

(52) U.S. Cl. 422/159; 702/27; 424/1.11

(57) ABSTRACT

A system and method for manufacturing radiopharmaceuticals uses only pressure to transfer radioactive reactants from a reactant vessel to purification and storage, thereby preventing radioactive contamination of manufacturing components.

### PRIORITY REFERENCE TO PRIOR APPLICATIONS

This application claims benefit of and incorporates by reference patent application Ser. No. 60/576,006, entitled “Apparatus And Method For Preventing Radioactive Contamination Of Radiopharmaceutical Manufacturing Components,” filed on Jun. 1, 2004, by inventor Ahmad Najafi.

### TECHNICAL FIELD

This invention relates generally to radiopharmaceutical manufacturing, and more particularly, but not exclusively, provides a system and method for preventing radioactive contamination of radiopharmaceutical manufacturing components.

### BACKGROUND

Positron Emission Tomography (PET), which emerged as the most sensitive technique for detection of any neuronal or cardiac cell damage, has now become a leading technique for early detection of many different malignancies. PET is also used for post-operative evaluation of cancer patients. This technique requires production of many Positron emitting radiopharmaceuticals including F-18-Fluorodeoxyglucose (FDG). FDG is by far the most produced radiopharmaceutical for PET scanning, since it is used for cancer detection, which is now mostly reimbursable by insurance companies. Other radiopharmaceuticals are needed and used for local neuronal or cardiac cell evaluations. F-18-6-fluoro-L-dopa (6FD) is used in conjunction with this technique for measurement of neuronal dopamine synthesis, which is the leading cause of Parkinson disease.

All of the Radiopharmaceuticals used for PET need to be prepared either automatically or by remote control due to high radiation levels. There are many automatic synthesizers available in the market primarily for FDG synthesis, including those made by GE and CTI. All of these automated synthesizers use pressure to add reagent(s) (usually an organic compound dissolved in an organic solvent) through a 1/16 inch Teflon tubing into a reaction vessel (usually a few milliliters glass test tube). After addition of all reagents, at the completion of the reaction, reactants (usually a highly radioactive organic compound(s) contaminated with radioactive gases dissolved in an organic solvent) transferred by applying vacuum into a syringe or a vessel (normally made out of glass, about few milliliters), and then pushed by pressure (normally using an inert gas such as nitrogen about 20 PSI) through an activation of a solenoid three way valve to the next place (reaction vessel). As a result many of the parts in these synthesizers that can stay non-radioactive need to be shielded because of radioactive contamination and potential leakage of radioactive gases through vacuum. This makes the synthesizers unnecessarily big and complicated, particularly when the shielded space in these laboratories are limited. In addition, for production of more complicated radiopharmaceuticals such as 6FD where there are far more reactants and steps it is very difficult to shield everything. In addition, accessibility to solvents/reagents during the production run is essential.

Accordingly, a new system and method are needed that prevent unnecessary contamination chemistry module component during radiopharmaceutical production.

### SUMMARY

A system and method for manufacturing radiopharmaceuticals uses only pressure to transfer radioactive reactants during radiopharmaceutical production, and final delivery to the final product vial, thereby preventing radioactive contamination of manufacturing components.

In an embodiment of the invention, the system comprises a gas inlet and a reaction vessel. The gas inlet injects gas (e.g., nitrogen) into the reaction vessel to transfer a radiopharmaceutical out of the reaction vessel.

In an embodiment of the invention, the method comprises: transferring reagents into a reaction vessel via pressure from a gas inlet; and transferring a radiopharmaceutical out of the reaction vessel via pressure from the gas inlet.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a block diagram illustrating a radiopharmaceutical manufacturing system according to an embodiment of the invention;

FIG. 2 is a diagram illustrating the system of FIG. 1 in more detail;

FIG. 3 is a diagram illustrating a computer of the system of FIG. 1;

FIG. 4 is a diagram illustrating a persistent memory of the computer of FIG. 1;

FIG. 5 is a flowchart illustrating a method of generating a radiopharmaceutical without radioactive contamination of radiopharmaceutical components; and

FIG. 6 is a diagram illustrating the manufacture of F-18 6-Flouoro-L-dopa.

### DETAILED DESCRIPTION OF THE ILLUSTRATED EMBODIMENTS

The following description is provided to enable any person having ordinary skill in the art to make and use the invention, and is provided in the context of a particular application and its requirements. Various modifications to the embodiments will be readily apparent to those skilled in the art, and the principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

Embodiments of the invention provide a system and method that use only pressure to add or transfer reagents and/or reactants from place to place in synthesizers. Therefore, these potentially non-radioactive parts will stay non-radioactive throughout the process since there is no vacuum used. As a result synthesizers can be made smaller; less complicated, and easier to place them in a shielded area.

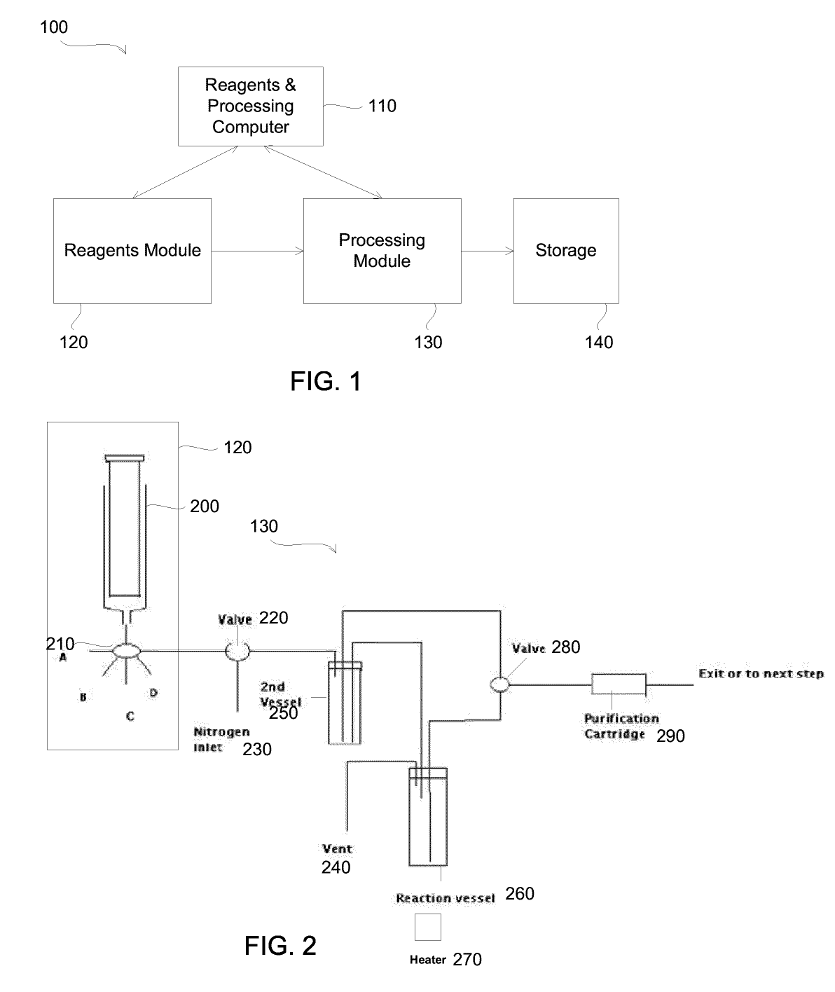

FIG. 1 is a block diagram illustrating a radiopharmaceutical manufacturing system 100 according to an embodiment of the invention. The system 100 comprises a reagents and processing computer communicatively coupled to a reagents module 120 and a processing module 130. The reagents module 120 is coupled to the processing module 130, which is coupled to storage 140. The computer 110, the reagents module 120 and the processing module 130 will be discussed in further detail below. The reagents module 120 holds non-radioactive reagents used in the manufacturing of radiopharmaceuticals. The processing module 130 manufactures radiopharmaceuticals using reagents from the reagents module 120. The storage 140 stores reactants (e.g., radipharmaceuticals) generated by the processing module 130. The computer 110 controls the operation of the reagents module 120 and the processing module 130, such as the transfer of reagents and reactants between the components.

FIG. 2 is a diagram illustrating the system 100 of FIG. 1 in more detail. The reagents module 120 comprises a syringe 200 coupled to a valve 210, which is coupled to reagent stores A, B, C, and D. It will be appreciated by one of ordinary skill in the art that the reagents module 120 can include additional or fewer reagent stores. Further, the reagent stores can include identical or different reagents in each store.

The processing module 130 is coupled to the valve 210 via a valve 220. Coupled to the valve 220 is a nitrogen inlet 230. In an embodiment of the invention, all inert gases besides nitrogen can be used, e.g., Helium, Argon, etc. The valve 220 is also coupled to a secondary vessel 250, which is coupled to a reaction vessel 260 via two or more lines. A first line is coupled directly to the reaction vessel 260 while a second is coupled to the reaction vessel 260 via a valve 280 (e.g., a 3-way solenoid valve). The reaction vessel 260 is coupled to a vent 240 and a heater 270. The valve 280 is coupled to a purification cartridge 290, which is coupled to the storage 140 (FIG. 1). The computer 110 is also communicatively coupled to the syringe 200, the nitrogen inlet 230, the valves 210, 220 and 280, the vent 240, and the heater 270.

During operation of the system 100, the computer 110 causes the valve 210, which can include a multi-way solenoid valve to open to one or more reagent stores A-D and the syringe 200. The computer 110 then causes the syringe 200 to suction out one or more reagents (either one at a time or in conjunction) from the stores A-D. The computer 110 then causes the valve 210 to close to the reagent stores A-D and open to the valve 220. The computer 110 also opens the valve 220 to the secondary vessel 250 and to the valve 210. The computer 110 then causes the syringe 200 to expel the reagents (one at a time or in conjunction) to the secondary vessel 250. At the secondary vessel 250, one or more reagents can react to generate one or more intermediate products.

The computer 110 then opens the valve 220 to the nitrogen inlet 230 and the secondary vessel 250 and causes the nitrogen inlet 230 to pump nitrogen into the secondary vessel 250, which causes the reagents and/or products in the secondary vessel 250 to transfer to the reaction vessel 260 via the two lines (the valve 280 is open except to the purification cartridge 290 at this point). Heat is then applied to the reaction vessel 260 via the heater 270 (as controlled by the computer 110). The computer 110 opens the vent 240 to release excess pressure and/or enable the evaporation of solvents or other unwanted products from the reaction in the reaction vessel 260. After the reaction is complete, the reactants in the reaction vessel 260 are radioactive and the computer 110 causes the nitrogen inlet to inject more nitrogen into the secondary vessel 250. The valve 280 is closed to the secondary vessel 250 and opened to the purification cartridge 290. The nitrogen then transfers to the reaction vessel 260, at which point the vent 240 is closed. The nitrogen causes the reactants to transfer to the purification cartridge 290, which purifies the reactants by getting rid of unwanted byproducts. Excess nitrogen can be vented out. The wanted reactant(s) is then transferred to storage 140 or another processing module 130 for further manufacturing steps.

In an embodiment of the invention, the reagent stores A, B, C, and D contain solvents such as ether, acetonitrile, etc., and/or reagents such as sodium borohydride, thionyl bromide, etc. The syringe 200 can include a Glass syringe of about 1 to about 30 mL. The secondary vessel 250 can have a volume of about 1 to about 10 mL. The reaction vessel 260 is about 10 mL. The reaction in the reaction vessel 260 can include isotope exchange, reduction, bromination, etc. Nitrogen pressure can be adjusted by computer to different levels from about 0 to about 100 PSI. The heater 270 can operate at about 100 to 400 F depending on the radiopharmaceutical being generated (e.g., 280 F for isotopic exchange). Reactants purified can include F-18 6-flouorovetraldehyde, f-18 6-flouoro-3,4-methoxy-benzylbromide, etc.

Accordingly, no suction is used to transfer radioactive reactants backwards into the system as in conventional systems. As such, any stages before the reaction vessel 260 remain non-radioactive and therefore do not require shielding, thereby reducing the space required, reducing complexity, and reducing costs.

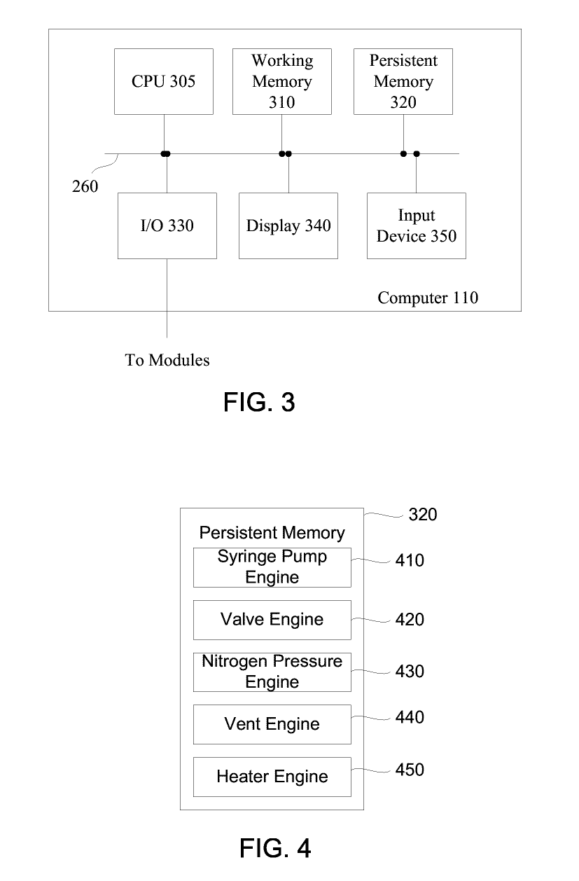

FIG. 3 is a diagram illustrating the computer 110 of the system 100. The example computer 110 includes a central processing unit (CPU) 305; working memory 310; persistent memory 320; input/output (I/O) interface 330; display 340; and input device 350, all communicatively coupled to each other via a bus 360. The CPU 305 may include an INTEL PENTIUM microprocessor, a Motorola POWERPC microprocessor, or any other processor capable to execute software stored in the persistent memory 320. The working memory 310 may include random access memory (RAM) or any other type of read/write memory devices or combination of memory devices. The persistent memory 320 may include a hard drive, read only memory (ROM) or any other type of memory device or combination of memory devices that can retain data after the example computer 300 is shut off. The I/O interface 330 is communicatively coupled, via wired or wireless techniques, to the modules 120 and 130. The display 340 may include a flat panel display, cathode ray tube display, or any other display device. The input device 350, which is optional like other components of the invention, may include a keyboard, mouse, or other device for inputting data, or a combination of devices for inputting data.

One skilled in the art will recognize that the computer 110 may also include additional devices, such as network connections, additional memory, additional processors, LANs, input/output lines for transferring information across a hardware channel, the Internet or an intranet, etc. One skilled in the art will also recognize that the programs and data may be received by and stored in the system in alternative ways. Further, in an embodiment of the invention, an ASIC is used in placed of the computer 110.

FIG. 4 is a diagram illustrating the persistent memory 320 of the computer 110. The memory 320 comprises a syringe pump engine 410, a valve engine 420, a nitrogen pressure engine 430, a vent engine 440, and a heater engine 450. In another embodiment of the invention, the engines 410-450 can be implemented in circuitry or with other techniques.

The syringe pump engine 410 is communicatively coupled to the syringe 200 and causes the syringe 200 to suck reagents from the reagents stores 210 one at a time or in conjunction from the reagent stores 210. The syringe pump engine 410 also causes the syringe 200 to expel the reagents into the secondary vessel 250. The valve engine 420 is communicatively coupled to the valves 210, 220, and 280 and causes the valves 210, 220 and 280 and open and close according to radiopharmaceutical manufacturing stage. The nitrogen pressure engine 430 is communicatively coupled to the nitrogen inlet 230 and causes nitrogen (or other gas) to enter the processing module 130 to cause the transfer and reagents and reactants from the secondary vessel 250 to the reaction vessel 260 and from the reaction vessel 260 through the purification cartridge 290 to the storage 140.

The vent engine 440 is communicatively coupled to the vent 240 and causes the vent 240 to open when a reaction is occurring in the reaction vessel 260 and to close when reagents are transferred into the vessel 260 and when reactants are being transferred out of the vessel 260. The heater engine 450 causes the heater 270 to apply heat to the reaction vessel 260 when the reagents are in the reaction vessel 260.

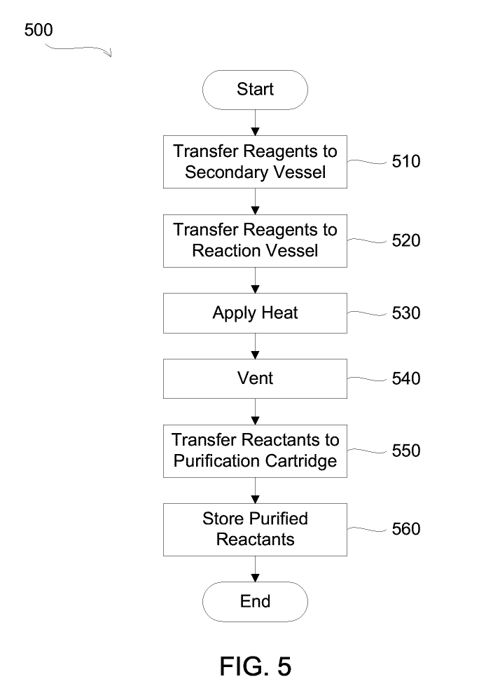

FIG. 5 is a flowchart illustrating a method 500 of generating a radiopharmaceutical without radioactive contamination of radiopharmaceutical components. In an embodiment of the invention, the system 100 implements the method 500. First, reagents are transferred (510) to the secondary vessel 250 by the syringe 200 sucking them from reagent stores A-D and then expelling them through the valves 210 and 220. The reagents are then transferred (520) to the reaction vessel 260 by inserting nitrogen into the secondary vessel 250 from the nitrogen inlet 230. Heat is then applied (530) to the reaction vessel 260 to cause the reagents to react and the vent is opened (540) to enable solvents to evaporate out of the reaction vessel 260 and/or to relieve pressure in the reaction vessel 260. The reactants are then transferred (550) to the purification cartridge where unwanted byproducts are removed from the reactants. The purified reactant(s) are then stored (560) in the storage 140. The method 500 then ends.

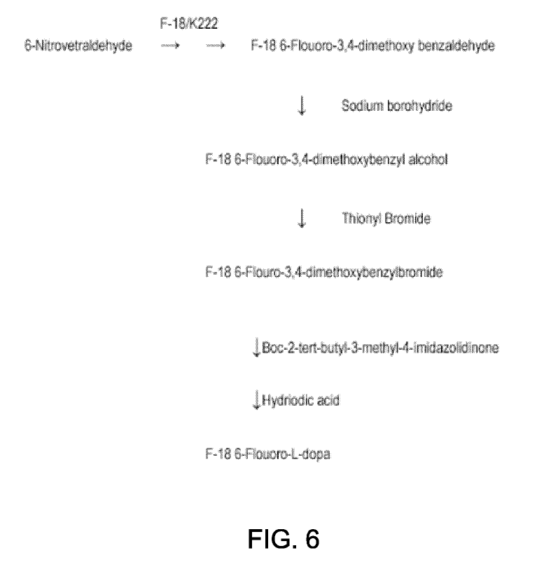

FIG. 6 is a diagram illustrating the manufacture of F18 6-Flouoro-L-dopa using an embodiment of the invention. The details of the manufacture can be found in the Journal of Nuclear Medicine and Biology, Volume 22, Number 3, Page 395 (1995).

The foregoing description of the illustrated embodiments of the present invention is by way of example only, and other variations and modifications of the above-described embodiments and methods are possible in light of the foregoing teaching. For example, other gases can be used besides nitrogen. Although the engines are being described as separate and distinct, one skilled in the art will recognize that these engines may be a part of an integral site, may each include portions of multiple engines, or may include combinations of single and multiple engines. Further, components of this invention may be implemented using a programmed general purpose digital computer, using application specific integrated circuits, or using a network of interconnected conventional components and circuits. Connections may be wired, wireless, modem, etc. The embodiments described herein are not intended to be exhaustive or limiting. The present invention is limited only by the following claims.

1. A radiopharmaceutical manufacturing system, comprising:
a gas inlet; and
a reaction vessel,
wherein the gas inlet is capable of injecting gas into the reaction vessel to transfer a radiopharmaceutical out of the reaction vessel.

2. The system of claim 1, wherein the gas includes nitrogen.

3. The system of claim 1, further comprising a syringe coupled to reagent stores via a first valve, and wherein the first valve is coupled to a second valve coupled to the gas inlet.

4. The system of claim 3, further comprising a secondary vessel coupled to the reaction vessel and the second valve.

5. The system of claim 4, wherein the secondary vessel is coupled to the reaction vessel by a first and a second line, wherein the second line is coupled to the reaction vessel via a third valve.

6. The system of claim 5, further comprising a purification cartridge coupled to the reaction vessel via the third valve.

7. The system of claim 5, further comprising a computer communicatively coupled to the first, second and third valves, the gas inlet, and the syringe.

8. A radiopharmaceutical manufacturing method comprising:
transferring reagents into a reaction vessel via pressure from a gas inlet;
applying heat to the reaction vessel; and
transferring a radiopharmaceutical out of the reaction vessel via pressure from the gas inlet.

9. The method of claim 8, wherein gas from the gas inlet includes nitrogen.

10. The method of claim 8, wherein a syringe is coupled to reagent stores via a first valve, and wherein the first valve is coupled to a second valve coupled to the gas inlet, wherein the method further comprises transferring reagents from the reagent stores into the syringe via suction.

11. The method of claim 10, wherein a secondary vessel is coupled to the reaction vessel and the second valve and the method further comprises transferring reagents from the secondary vessel to the reaction vessel via pressure from the gas inlet.

12. The method of claim 11, wherein the secondary vessel is coupled to the reaction vessel by a first and a second line, wherein the second line is coupled to the reaction vessel via a third valve.

13. The method of claim 12, wherein a purification cartridge coupled to the reaction vessel via the third valve and wherein the method further comprises purifying the radioactive chemicals of unwanted byproducts with the purification cartridge.

14. A computer readable medium having stored thereon instructions to cause a computer to execute a radiopharmaceutical manufacturing method, the method comprising:
transferring reagents into a reaction vessel via pressure from a gas inlet;
applying heat to the reaction vessel; and
transferring a radiopharmaceutical out of the reaction vessel via pressure from the gas inlet.

15. A radiopharmaceutical manufacturing system, comprising:
means for transferring reagents into a reaction vessel via pressure from a gas inlet;
means for applying heat to the reaction vessel; and
means for transferring a radiopharmaceutical out of the reaction vessel via pressure from the gas inlet.
